(19) United States
(12) Patent Application Publication
(10) Pub. No.: US 2020/0281918 A1
(43) Pub. Date: Sep. 10, 2020

(54) CALCIUM RELEASE-ACTIVATED CALCIUM CHANNEL MODULATORS FOR TREATING HEMATOLOGICAL AND SOLID CANCERS

(71) Applicant: Rhizen Pharmaceuticals SA, La Chaux-de-Fonds (CH)

(72) Inventors: Srikant Viswanadha, Hyderabad (IN); Swaroop Kumar Kumar Venkata Sat Vekalanka, La Chaux-de-Fonds (CH)

(21) Appl. No.: 16/759,629

(22) PCT Filed: Oct. 29, 2018
(86) PCT No.: PCT/IB2018/058461

§ 371 Date: Apr. 27, 2020

(30) Foreign Application Priority Data
Oct. 30, 2017 (IN) 201741038447

(51) Int. Cl. A61K 31/4709; A61K 9/00; A61K 45/06; A61P 35/00

(52) U.S. Cl. CPC A61K 31/4709; A61P 35/00; A61K 45/06; A61K 9/0053

(57) ABSTRACT

The present invention relates to the use of a calcium release-activated calcium (CRAC) channel modulator, such as N-[4-(3,5-dicyclopropyl-1H-pyrazol-1-yl)phenyl]-2-(quinolin-6-yl)acetamide (Compound (A)) or a pharmaceutically acceptable salt thereof, or a pharmaceutical composition containing such a CRAC channel modulator for the treatment of haematological and solid cancers.

The present invention claims the benefit of Indian Provisional Application No. 201741038447, filed 30 Oct. 2017, which is hereby incorporated by reference in its entirety.

### FIELD OF THE INVENTION

The present invention relates to the use of a calcium release-activated calcium (CRAC) channel modulator, such as N-[4-(3,5-dicyclopropyl-1H-pyrazol-1-yl)phenyl]-2-(quinolin-6-yl)acetamide (Compound (A)) or a pharmaceutically acceptable salt thereof, or a pharmaceutical composition containing such a CRAC channel modulator for the treatment of haematological and solid cancers.

### BACKGROUND OF THE INVENTION

A solid tumor is an abnormal mass of tissue that usually does not contain cysts or liquid areas. Solid tumors may be benign (not cancerous), or malignant (cancerous). Different types of solid tumors are named for the type of cells that form them. Examples of solid tumors include sarcomas, carcinomas, and lymphomas. Leukemias (cancers of the blood) generally do not form solid tumors.

Two major types of solid tumors are sarcomas and carcinomas. Sarcomas are tumors in a blood vessel, bone, fat tissue, ligament, lymph vessel, muscle or tendon. There are many types of sarcomas. They include Ewing sarcoma and osteosarcoma, which are bone cancer sarcomas. Rhabdomyosarcoma is a soft tissue sarcoma found in muscles.

Carcinomas are tumors that form in epithelial cells. Epithelial cells are found in the skin, glands and the linings of organs such as the bladder, ureter and parts of the kidneys. One common carcinoma is adrenocortical carcinoma. This is when a tumor develops in one or both adrenal glands, located above each kidney. See https://www.stjude.org/treatment/disease/solid-tumors/what-is-solid-tumor.html.

Hematologic malignancies are forms of cancer that begin in the cells of blood-forming tissue, such as the bone marrow, or in the cells of the immune system. Examples of hematologic cancer are acute and chronic leukemias, lymphomas, multiple myeloma and myelodysplastic syndromes. While uncommon in solid tumors, chromosomal translocations are a common cause of these diseases. This commonly leads to a different approach in diagnosis and treatment of haematological malignancies. See https://www.omicsonline.org/scholarly/hematologic-malignancies-journals-articles-ppts-list.php.

During the past several decades, substantial progress has been made in the treatment of hematologic malignancies, particularly in some subgroups of patients. Today, cure is attainable for patients with Hodgkin's disease and a considerable proportion of patients with high-grade non-Hodgkin's lymphoma. Prognosis is improving in patients with acute promyelocytic leukemia and, to some extent, those with acute lymphoblastic and myeloid leukemias. However, the majority of patients who suffer from a hematologic malignancy live with incurable disease. In chronic lymphocytic leukemia (CLL), outside the setting of a clinical trial, it is advisable to postpone treatment until the manifestation of clinical symptoms. It is yet to be determined whether treatment strategies based on new prognostic parameters such as cytogenetics can change the course of disease. In indolent lymphomas, cure is not attainable for the vast majority of patients and the median survival of 9 to 10 years has remained unchanged for several decades. Finally, regardless of underlying malignancy and prognosis, the preservation of quality of life is of major consideration in the setting of hematologic malignancies. See Voliotis et. al., Semin. Oncol., 2002 June; 29(3 Suppl. 8):30-9.

Despite some progress made in the treatment of hematological and solid cancers, challenges remain in terms of the treatment, side effects and desired clinical benefits from small molecule inhibitors. Accordingly, there still remains an unmet need for drugs for the treatment and/or amelioration of hematological and solid cancers.

### SUMMARY OF THE INVENTION

In one aspect, the present invention relates to the use of a calcium release-activated calcium (CRAC) channel modulator, such as a CRAC channel inhibitor, for treating haematological and solid cancers.

The inventors surprisingly found that the CRAC channel inhibitor N-(4-(3,5-dicyclopropyl-1H-pyrazol-1-yl)phenyl)-2-(quinolin-6-yl)acetamide (Compound (A), shown below) exhibits excellent activity against haematological and solid cancers.

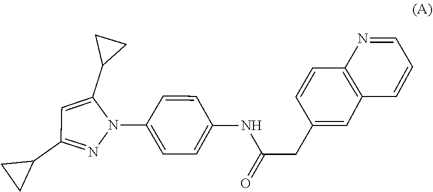 [SMILES: O=C(Cc1ccc2ncccc2c1)Nc1ccc(-n2nc(C3CC3)cc2C2CC2)cc1]

One embodiment is the use of a CRAC channel modulator, such as a CRAC channel inhibitor, for the treatment of a haematological or solid cancer. A preferred embodiment is the use of the CRAC channel inhibitor Compound (A) or a pharmaceutically acceptable salt thereof for the treatment of a haematological cancer or a solid cancer.

The CRAC channel modulator may be administered as a front-line therapy or as a relapsed-refractory therapy for the treatment of a haematological cancer.

The CRAC channel modulator may be administered as a first-line therapy or as a second-line therapy or as a subsequent treatment for the treatment of a solid cancer.

Another embodiment is a method of treating a haematological or solid cancer in a subject (preferably a human subject) comprising administering to the subject an effective amount of a CRAC channel modulator. In one embodiment, the CRAC channel modulator is a CRAC channel inhibitor.

A preferred embodiment is a method of treating a haematological or solid cancer in a subject (preferably a human subject) comprising administering to the subject (preferably a human subject) an effective amount of Compound (A) or a pharmaceutically acceptable salt thereof.

Yet another embodiment is a method of modulating CRAC channels in a subject (preferably a human subject) suffering from a haematological or solid cancer by administering to the subject an effective amount of a CRAC channel modulator. In a preferred embodiment, the CRAC channel modulator is Compound (A) or a pharmaceutically acceptable salt thereof.

An object of the present invention relates to the uses described herein for the treatment of a subject, in particular of a human subject.

An object of the present invention is the use of Compound (A) or a pharmaceutically acceptable salt thereof for the preparation of a medicament intended for the treatment of a haematological or solid cancer.

Another object of the present invention is the use of Compound (A) or a pharmaceutically acceptable salt thereof for the preparation of a medicament intended for the treatment of a haematological or solid cancer, where the medicament is administered orally.

In a preferred embodiment, the haematological cancer is leukemia, lymphoma or multiple myeloma.

In another embodiment, the solid cancer is sarcoma, carcinoma or lymphoma.

In a preferred embodiment, Compound (A) is administered as a hydrochloric acid salt of Compound (A). For example, Compound (A) may be administered as N-(4-(3,5-dicyclopropyl-1H-pyrazol-1-yl)phenyl)-2-(quinolin-6-yl)acetamide hydrochloride.

The CRAC channel modulator, such as Compound (A) or a pharmaceutically acceptable salt thereof, can be administered to the subject by the oral route, the intravenous route, the intramuscular route, or the intraperitoneal route. In one preferred embodiment, the CRAC channel modulator is administered orally.

In one embodiment, the CRAC channel modulator, such as Compound (A) or a pharmaceutically acceptable salt thereof, is administered as a front-line therapy for a haematological cancer.

In another embodiment, the CRAC channel modulator, such as Compound (A) or a pharmaceutically acceptable salt thereof, is administered as a relapsed-refractory therapy for a haematological cancer.

In one embodiment, the CRAC channel modulator, such as Compound (A) or a pharmaceutically acceptable salt thereof, is administered as a first-line therapy for a solid cancer.

In another embodiment, the CRAC channel modulator, such as Compound (A) or a pharmaceutically acceptable salt thereof, is administered as a non-resectable solid cancer therapy.

In yet another embodiment, in any of the uses of the CRAC channel modulator and methods described herein, the CRAC channel modulator is used in combination (administered together or sequentially) with an anti-cancer treatment, one or more cytostatic, cytotoxic or anticancer agents, targeted therapy, or any combination or any of the foregoing.

Suitable anti-cancer treatments include, e.g., radiation therapy. Suitable cytostatic, cytotoxic and anticancer agents include, but are not limited to, DNA interactive agents, such as cisplatin or doxorubicin; topoisomerase II inhibitors, such as etoposide; topoisomerase I inhibitors such as CPT-11 or topotecan; tubulin interacting agents, such as paclitaxel, docetaxel or the epothilones (for example, ixabepilone), either naturally occurring or synthetic; hormonal agents, such as tamoxifen; thymidilate synthase inhibitors, such as 5-fluorouracil; and anti-metabolites, such as methotrexate, other tyrosine kinase inhibitors such as gefitinib (marketed as Iressac) and erlotinib (also known as OSI-774); angiogenesis inhibitors; EGF inhibitors; VEGF inhibitors; CDK inhibitors; SRC inhibitors; c-Kit inhibitors; Her1/2 inhibitors and monoclonal antibodies directed against growth factor receptors such as erbitux (EGF) and herceptin (Her2), and other protein kinase modulators.

Yet another embodiment is Compound (A) or a pharmaceutically acceptable salt thereof suitable for use in the front-line therapy of a haematological cancer.

Yet another embodiment is Compound (A) or a pharmaceutically acceptable salt thereof suitable for use in the relapsed-refractory therapy of a haematological cancer.

Yet another embodiment is Compound (A) or a pharmaceutically acceptable salt thereof suitable for use in the first-line therapy of a solid cancer.

Yet another embodiment is Compound (A) or a pharmaceutically acceptable salt thereof suitable for use in non-resectable solid cancer therapy (i.e., for treatment of non-resectable solid cancer).

Yet another embodiment is a pharmaceutical composition for treating a haematological or solid cancer comprising a CRAC channel modulator, such as a CRAC channel inhibitor (preferably Compound (A) or a pharmaceutically acceptable salt thereof), and optionally one or more pharmaceutically acceptable carriers or excipients.

In a preferred embodiment, the CRAC channel modulator is a hydrochloride (HCl) salt of Compound (A).

In one embodiment, the pharmaceutical composition further comprises one or more cytostatic, cytotoxic or anticancer agents.

In one embodiment, the pharmaceutical composition is useful in combination with one or more anti-cancer treatments, one or more cytostatic, cytotoxic or anticancer agents, targeted therapy, or any combination or any of the foregoing. The CRAC channel modulator may be used together or sequentially with one or more anti-cancer treatments one or more cytostatic, cytotoxic or anticancer agents, targeted therapy, or any combination or any of the foregoing.

In one preferred embodiment, the pharmaceutical composition is suitable for oral administration. In a more preferred embodiment, the CRAC channel modulator in the pharmaceutical composition for oral administration is a hydrochloride salt of Compound (A).

In another embodiment, Compound (A) or a pharmaceutically acceptable salt thereof is administered at a dose of about 25 to about 1000 mg, such as a dose of about 25 to about 800 mg, about 25 to about 600 mg, about 25 to about 400 mg, or about 25 to about 200 mg.

In yet another embodiment, Compound (A) or a pharmaceutically acceptable salt thereof is administered at a dose of about 50 to about 1000 mg, such as a dose of about 50 to about 800 mg, about 50 to about 600 mg, about 50 to about 400 mg, or about 50 to about 200 mg.

In another embodiment, Compound (A) or a pharmaceutically acceptable salt thereof is administered at a dose of about 100 to about 1000 mg, such as a dose of about 100 to about 800 mg, about 100 to about 600 mg, about 100 to about 400 mg, or about 100 to about 200 mg.

In another embodiment, Compound (A) or a pharmaceutically acceptable salt thereof is administered at a dose of about 25 to about 1000 mg per day, such as a dose of about 50 to about 500-mg per day or a dose of about 100 to about 400 mg per day.

Compound (A) or a pharmaceutically acceptable salt thereof may be administered as a single dose or in divided doses.

In another embodiment, Compound (A) or a pharmaceutically acceptable salt thereof, is administered once daily. In yet another embodiment, Compound (A) or a pharmaceutically acceptable salt thereof is administered twice daily.

In the uses and methods described herein, the subject can be a human subject suffering from relapsed haematological cancer, refractory haematological cancer, or relapsed-refractory haematological cancer.

In the uses and methods described herein, the subject can be a human subject suffering from relapsed solid cancer, refractory solid cancer, or relapsed-refractory solid cancer.

### BRIEF DESCRIPTION OF THE FIGURES

FIG. 1 is a bar graph showing the effect of Compound (A) at 10, 100, 1,000, 3,000, and 10,000 nM and a blank on the induction of Caspase 3 activity in lung cancer cells.

FIG. 2 is a bar graph showing the effect of Compound (A) at 10, 100, 300, 1,000, and 10,000 nM on migration in A549 cells as measured by inhibition in the scratch-wound assay of Example 2.

FIG. 3 is a bar graph showing the anti-proliferative effect (GI₅₀ values measured in nM) of Compound (A) in various cancer cell lines.

FIG. 4 shows the inhibitory effect of Compound (A) on NFkB, Phospho-mTOR and phosphor-S6 in Jurkat cells.

FIG. 5 is a graph showing the anti-tumor effects of Compound (A) and taxol in a NCI-H460 human non-small cell lung cancer xenograft model in mice.

### DETAIL DESCRIPTION OF THE INVENTION

### Definitions

Unless defined otherwise, all technical and scientific terms used herein have the same meaning as is commonly understood in the field to which the subject matter belongs. In the event that there is a plurality of definitions for terms herein, those in this section prevail. Where reference is made to a URL or other such identifier or address, it is understood that such identifiers generally change and particular information on the internet comes and goes, but equivalent information is found by searching the internet. Reference thereto evidences the availability and public dissemination of such information.

It is to be understood that the foregoing general description and the following detailed description are exemplary and explanatory only and are not restrictive of any subject matter.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Definition of standard chemistry and molecular biology terms are found in reference works including, but not limited to, Carey and Sundberg “ADVANCED ORGANIC CHEMISTRY 4th edition” Vols. A (2000) and B (2001), Plenum Press, New York and “MOLECULAR BIOLOGY OF THE CELL 5^{th} edition” (2007), Garland Science, New York. Unless otherwise indicated, conventional methods of mass spectroscopy, NMR, HPLC, protein chemistry, biochemistry, recombinant DNA techniques and pharmacology are contemplated within the scope of the embodiments disclosed herein.

Unless specific definitions are provided, the nomenclature employed in connection with, and the laboratory procedures and techniques of, analytical chemistry, and medicinal and pharmaceutical chemistry described herein are those generally used. In some embodiments, standard techniques are used for chemical analyses, pharmaceutical preparation, formulation, and delivery, and treatment of patients. In other embodiments, standard techniques are used for recombinant DNA, oligonucleotide synthesis, and tissue culture and transformation (e.g., electroporation, lipofection). In certain embodiments, reactions and purification techniques are performed e.g., using kits of manufacturer's specifications or as described herein. The foregoing techniques and procedures are generally performed of conventional methods and as described in various general and more specific references that are cited and discussed throughout the present specification.

Additionally, the CRAC channel modulators described herein, including Compound (A) and pharmaceutically acceptable salts thereof, include compounds which differ only in the presence of one or more isotopically enriched atoms, for example, replacement of hydrogen with deuterium.

The term “subject” or “patient” encompasses mammals and non-mammals Examples of mammals include, but are not limited to, any member of the Mammalian class: humans, non-human primates such as chimpanzees, and other apes and monkey species; farm animals such as cattle, horses, sheep, goats, and swine; domestic animals such as rabbits, dogs, and cats; and laboratory animals including rodents, such as rats, mice and guinea pigs. Examples of non-mammals include, but are not limited to, birds, fish and the like. In one preferred embodiment of the methods, uses, and compositions provided herein, the mammal is a human.

The terms “treat,” “treating” or “treatment,” as used herein, include alleviating, abating or ameliorating a disease, disorder or condition symptoms, preventing additional symptoms, ameliorating or preventing the underlying causes of symptoms, inhibiting the disease, disorder or condition, e.g., arresting the development of the disease, disorder or condition, relieving the disease, disorder or condition, causing regression of the disease, disorder or condition, relieving a condition caused by the disease, disorder or condition, or stopping the symptoms of the disease, disorder or condition either prophylactically and/or therapeutically.

The term “front-line therapy” refers to the first treatment given for a disease. It is often part of a standard set of treatments, such as surgery followed by chemotherapy and radiation. When used by itself, front-line therapy is the one accepted as the best treatment. If it doesn't cure the disease or it causes severe side effects, other treatment may be added or used instead. It is also called induction therapy, primary therapy, and primary treatment.

The term “relapsed” refers to disease that reappears or grows again after a period of remission.

The term “refractory” is used to describe when the cancer does not respond to treatment (meaning that the cancer cells continue to grow) or when the response to treatment does not last very long.

The term “first-line therapy” refers to the first treatment given for a disease. It is often part of a standard set of treatments, such as surgery followed by chemotherapy and radiation. When used by itself, first-line therapy is the one accepted as the best treatment. If it doesn't cure the disease or it causes severe side effects, other treatment may be added or used instead. It is also called induction therapy, primary therapy, and primary treatment.

The term “second-line therapy” refers to a treatment that is given when initial treatment (first-line therapy) is not sufficiently effective, or stops being sufficiently effective.

The term “haematological cancer” refers to a cancer that begins in blood-forming tissue, such as the bone marrow, or in the cells of the immune system. Examples of haematological cancer include leukemia, lymphoma, and multiple myeloma. Hematological cancer is also called blood cancer.

The term “solid cancer” refers to an abnormal cellular growth in solid organs and forms malignant solid tumors. Different types of solid cancers are named for the type of cells that form them. Examples of solid cancers include sarcomas, carcinomas, and lymphomas.

As used herein, the term “target protein” refers to a protein or a portion of a protein capable of being bound by, or interacting with a compound described herein, such as a compound capable of modulating a STIM protein and/or an Orai protein. In certain embodiments, a target protein is a STIM protein. In other embodiments, a target protein is an Orai protein, and in yet other embodiments, the compound targets both STIM and Orai proteins.

The term “STIM protein” refers to any protein situated in the endoplasmic reticular or plasma membrane which activates an increase in rate of calcium flow into a cell by a CRAC channel. (STIM refers to a stromal interaction molecule.) As used herein, “STIM protein” includes, but is not limited to, mammalian STIM-1, such as human and rodent (e.g., mouse) STIM-1, Drosophila melanogaster D-STIM, C. elegans C-STIM, Anopheles gambiae STIM and mammalian STIM-2, such as human and rodent (e.g., mouse) STIM-2. As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum).

It will be appreciated by “activate” or “activation” it is meant the capacity of a STIM protein to up-regulate, stimulate, enhance or otherwise facilitate calcium flow into a cell by a CRAC channel. It is envisaged that cross-talk between the STIM protein and the CRAC channel may occur by either a direct or indirect molecular interaction. Suitably, the STIM protein is a transmembrane protein which is associated with, or in close proximity to, a CRAC channel.

It is known in the art that STIM1 is an essential component of CRAC channel activation. The present inventors have observed that STIM1 and STIM2 is expressed in certain ESCC cell lines. Moreover, CRACM1/Orai1 and CRACM3/Orai3 are excessively expressed in certain ESCC cell lines. Although not wishing to be bound by any particular theory, CRAC and STIM proteins potentially contribute to activation of proliferative pathways in ESCC cells in the following manner: (i) excessive dysregulation of STIM in ESCC cells results in incorrect plasma membrane accumulation of STIM and (ii) at the plasma membrane, STIM activates CRAC (by either a direct or indirect interaction), which results in excessive calcium influx into the cell and promotion of transcription, proliferation and invasiveness in ESCC cells. Hence, inhibition of the CRAC channel or the STIM pathway is an effective treatment for ESCC.

As used herein, an “Orai protein” includes Orai1 (SEQ ID NO: 1 as described in WO 07/081804), Orai2 (SEQ ID NO: 2 as described in WO 07/081804), or Orai3 (SEQ ID NO: 3 as described in WO 07/081804). Orai1 nucleic acid sequence corresponds to GenBank accession number NM-032790, Orai2 nucleic acid sequence corresponds to GenBank accession number BC069270 and Orai3 nucleic acid sequence corresponds to GenBank accession number NM-152288. As used herein, Orai refers to any one of the Orai genes, e.g., Orai1, Orai2, and Orai3 (see Table I of WO 07/081804). As described herein, such proteins have been identified as being involved in, participating in and/or providing for store-operated calcium entry or modulation thereof, cytoplasmic calcium buffering and/or modulation of calcium levels in or movement of calcium into, within or out of intracellular calcium stores (e.g., endoplasmic reticulum). In alternative embodiments, an Orai protein may be labelled with a tag molecule, by way of example only, an enzyme fragment, a protein (e.g. c-myc or other tag protein or fragment thereof), an enzyme tag, a fluorescent tag, a fluorophore tag, a chromophore tag, a Raman-activated tag, a chemiluminescent tag, a quantum dot marker, an antibody, a radioactive tag, or combination thereof.

The term “fragment” or “derivative” when referring to a protein (e.g. STIM, Orai) means proteins or polypeptides which retain essentially the same biological function or activity in at least one assay as the native protein(s). For example, the fragment or derivative of the referenced protein preferably maintains at least about 50% of the activity of the native protein, at least 75%, or at least about 95% of the activity of the native protein, as determined, e.g., by a calcium influx assay.

As used herein, “amelioration” refers to an improvement in a disease or condition or at least a partial relief of symptoms associated with a disease or condition. As used herein, amelioration of the symptoms of a particular disease, disorder or condition by administration of a particular compound or pharmaceutical composition refers to any lessening of severity, delay in onset, slowing of progression, or shortening of duration, whether permanent or temporary, lasting or transient that are attributed to or associated with administration of the compound or composition.

The term “modulate,” as used herein, means to interact with a target protein either directly or indirectly so as to alter the activity of the target protein, including, by way of example only, to inhibit the activity of the target, or to limit or reduce the activity of the target.

As used herein, the term “modulator” refers to a compound that alters an activity of a target (e.g., a target protein). For example, in some embodiments, a modulator causes an increase or decrease in the magnitude of a certain activity of a target compared to the magnitude of the activity in the absence of the modulator. In certain embodiments, a modulator is an inhibitor, which decreases the magnitude of one or more activities of a target, In certain embodiments, an inhibitor completely prevents one or more activities of a target.

As used herein, “modulation” with reference to intracellular calcium refers to any alteration or adjustment in intracellular calcium including but not limited to alteration of calcium concentration in the cytoplasm and/or intracellular calcium storage organelles, e.g., endoplasmic reticulum, or alteration of the kinetics of calcium fluxes into, out of and within cells. In aspect, modulation refers to reduction.

The terms “inhibits”, “inhibiting”, and “inhibitor” of SOC channel activity or CRAC channel activity, as used herein, refer to inhibition of store operated calcium channel activity or calcium release activated calcium channel activity.

The term “acceptable” with respect to a formulation, composition or ingredient, as used herein, means having no persistent detrimental effect on the general health of the subject being treated.

By “pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

Pharmaceutically acceptable salts forming part of this invention include salts derived from inorganic bases such as Li, Na, K, Ca, Mg, Fe, Cu, Zn, and Mn; salts of organic bases such as N,N′-diacetylethylenediamine, glucamine, triethylamine, choline, hydroxide, dicyclohexylamine, metformin, benzylamine, trialkylamine, thiamine, and the like; chiral bases like alkylphenylamine, glycinol, and phenyl glycinol, salts of natural amino acids such as glycine, alanine, valine, leucine, isoleucine, norleucine, tyrosine, cystine, cysteine, methionine, proline, hydroxy proline, histidine, omithine, lysine, arginine, and serine; quaternary ammonium salts of the compounds of invention with alkyl halides, and alkyl sulphates such as MeI and (Me)₂SO₄, non-natural amino acids such as D-isomers or substituted amino acids; guanidine, substituted guanidine wherein the substituents are selected from nitro, amino, alkyl, alkenyl, alkynyl, ammonium or substituted ammonium salts and aluminum salts. Salts may include acid addition salts where appropriate which are, sulphates, nitrates, phosphates, perchlorates, borates, hydrohalides, acetates, tartrates, maleates, citrates, fumarates, succinates, palmoates, methanesulphonates, benzoates, salicylates, benzenesulfonates, ascorbates, glycerophosphates, and ketoglutarates. Pharmaceutically acceptable solvates may be hydrates or comprise other solvents of crystallization such as alcohols.

The term “pharmaceutical composition” refers to a composition containing a CRAC channel modulator with one or more other chemical components, such as carriers, stabilizers, diluents, dispersing agents, suspending agents, thickening agents, and/or excipients.

The compounds (such as Compound (A) or a pharmaceutically acceptable salt thereof) and pharmaceutical compositions of the present invention can be administered by various routes of administration including, but not limited to, intravenous, oral, aerosol, parenteral, ophthalmic, pulmonary and topical administration.

The terms “effective amount” or “therapeutically effective amount,” as used herein, refer to a sufficient amount of an agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic uses is the amount of a compound of the present invention required to provide a clinically significant decrease in disease symptoms. In some embodiments, an appropriate “effective” amount in any individual case is determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “carrier,” as used herein, refers to relatively nontoxic chemical compounds or agents that facilitate the incorporation of a compound into cells or tissues.

The term “diluent” refers to chemical compounds that are used to dilute the compound of interest prior to delivery. In some embodiments, diluents are used to stabilize compounds because they provide a more stable environment. Salts dissolved in buffered solutions (which also provide pH control or maintenance) are utilized as diluents, including, but not limited to a phosphate buffered saline solution.

As used herein, “intracellular calcium” refers to calcium located in a cell without specification of a particular cellular location. In contrast, “cytosolic” or “cytoplasmic” with reference to calcium refers to calcium located in the cell cytoplasm.

As used herein, an effect on intracellular calcium is any alteration of any aspect of intracellular calcium, including but not limited to, an alteration in intracellular calcium levels and location and movement of calcium into, out of or within a cell or intracellular calcium store or organelle. For example, in some embodiments, an effect on intracellular calcium is an alteration of the properties, such as, for example, the kinetics, sensitivities, rate, amplitude, and electrophysiological characteristics, of calcium flux or movement that occurs in a cell or portion thereof. In some embodiments, an effect on intracellular calcium is an alteration in any intracellular calcium-modulating process, including, store-operated calcium entry, cytosolic calcium buffering, and calcium levels in or movement of calcium into, out of or within an intracellular calcium store. Any of these aspects are assessed in a variety of ways including, but not limited to, evaluation of calcium or other ion (particularly cation) levels, movement of calcium or other ion (particularly cation), fluctuations in calcium or other ion (particularly cation) levels, kinetics of calcium or other ion (particularly cation) fluxes and/or transport of calcium or other ion (particularly cation) through a membrane. An alteration is any such change that is statistically significant. Thus, for example, in some embodiments, if intracellular calcium in a test cell and a control cell is said to differ, such differences are a statistically significant difference.

Modulation of intracellular calcium is any alteration or adjustment in intracellular calcium including but not limited to alteration of calcium concentration or level in the cytoplasm and/or intracellular calcium storage organelles, e.g., endoplasmic reticulum, alteration in the movement of calcium into, out of and within a cell or intracellular calcium store or organelle, alteration in the location of calcium within a cell, and alteration of the kinetics, or other properties, of calcium fluxes into, out of and within cells. In some embodiments, intracellular calcium modulation involves alteration or adjustment, e.g. reduction or inhibition, of store-operated calcium entry, cytosolic calcium buffering, calcium levels in or movement of calcium into, out of or within an intracellular calcium store or organelle, and/or basal or resting cytosolic calcium levels. The modulation of intracellular calcium involves an alteration or adjustment in receptor-mediated ion (e.g., calcium) movement, second messenger-operated ion (e.g., calcium) movement, calcium influx into or efflux out of a cell, and/or ion (e.g., calcium) uptake into or release from intracellular compartments, including, for example, endosomes and lysosomes.

As used herein, “involved in,” with respect to the relationship between a protein and an aspect of intracellular calcium or intracellular calcium regulation means that when expression or activity of the protein in a cell is reduced, altered or eliminated, there is a concomitant or associated reduction, alteration or elimination of one or more aspects of intracellular calcium or intracellular calcium regulation. Such an alteration or reduction in expression or activity occurs by virtue of an alteration of expression of a gene encoding the protein or by altering the levels of the protein. A protein involved in an aspect of intracellular calcium, such as, for example, store-operated calcium entry, thus, are one that provides for or participates in an aspect of intracellular calcium or intracellular calcium regulation. For example, a protein that provides for store-operated calcium entry are a STIM protein and/or an Orai protein.

As used herein, a protein that is a component of a calcium channel is a protein that participates in multi-protein complex that forms the channel.

As used herein, “cation entry” or “calcium entry” into a cell refers to entry of cations, such as calcium, into an intracellular location, such as the cytoplasm of a cell or into the lumen of an intracellular organelle or storage site. Thus, in some embodiments, cation entry is, for example, the movement of cations into the cell cytoplasm from the extracellular medium or from an intracellular organelle or storage site, or the movement of cations into an intracellular organelle or storage site from the cytoplasm or extracellular medium. Movement of calcium into the cytoplasm from an intracellular organelle or storage site is also referred to as “calcium release” from the organelle or storage site.

As used herein, “immune cells” include cells of the immune system and cells that perform a function or activity in an immune response, such as, but not limited to, T-cells, B-cells, lymphocytes, macrophages, dendritic cells, neutrophils, eosinophils, basophils, mast cells, plasma cells, white blood cells, antigen presenting cells and natural killer cells.

“Store operated calcium entry” or “SOCE” refers to the mechanism by which release of calcium ions from intracellular stores is coordinated with ion influx across the plasma membrane.

Cellular calcium homeostasis is a result of the summation of regulatory systems involved in the control of intracellular calcium levels and movements. Cellular calcium homeostasis is achieved, at least in part, by calcium binding and by movement of calcium into and out of the cell across the plasma membrane and within the cell by movement of calcium across membranes of intracellular organelles including, for example, the endoplasmic reticulum, sarcoplasmic reticulum, mitochondria and endocytic organelles including endosomes and lysosomes.

Movement of calcium across cellular membranes is carried out by specialized proteins. For example, calcium from the extracellular space enters the cell through various calcium channels and a sodium/calcium exchanger and is actively extruded from the cell by calcium pumps and sodium/calcium exchangers. Calcium is also released from internal stores through inositol trisphosphate or ryanodine receptors and is likely taken up by these organelles by means of calcium pumps.

Calcium enters cells by any of several general classes of channels, including but not limited to, voltage-operated calcium (VOC) channels, store-operated calcium (SOC) channels, and sodium/calcium exchangers operating in reverse mode. VOC channels are activated by membrane depolarization and are found in excitable cells like nerve and muscle and are for the most part not found in nonexcitable cells. Under some conditions, Ca²⁺ also enters cells via Na⁺-Ca²⁺ exchangers operating in reverse mode.

Endocytosis provides another process by which cells take up calcium from the extracellular medium through endosomes. In addition, some cells, e.g., exocrine cells, release calcium via exocytosis.

Cytosolic calcium concentration is tightly regulated with resting levels usually estimated at approximately 0.1 μM in mammalian cells, whereas the extracellular calcium concentration is typically about 2 mM. This tight regulation facilitates transduction of signals into and within cells through transient calcium flux across the plasma membrane and membranes of intracellular organelles. There is a multiplicity of intracellular calcium transport and buffer systems in cells that serve to shape intracellular calcium signals and maintain the low resting cytoplasmic calcium concentration. In cells at rest, the principal components involved in maintaining basal calcium levels are calcium pumps and leaks in the endoplasmic reticulum and plasma membrane. Disturbance of resting cytosolic calcium levels effects transmission of such signals and give rise to defects in a number of cellular processes. For example, cell proliferation involves a prolonged calcium signalling sequence. Other cellular processes include, but are not limited to, secretion, signalling, and fertilization, involve calcium signalling.

Cell-surface receptors that activate phospholipase C(PLC) create cytosolic Ca²⁺ signals from intra- and extra-cellular sources. An initial transient rise of [Ca²⁺]i (intracellular calcium concentration) results from the release of Ca²⁺ from the endoplasmic reticulum (ER), which is triggered by the PLC product, inositol-1,4,5-trisphosphate (P3), opening IP3 receptors in the ER (Streb et al. Nature, 306, 67-69, 1983). A subsequent phase of sustained Ca²⁺ entry across the plasma membrane then ensues, through specialized store operated calcium (SOC) channels (in the case of immune cells the SOC channels are calcium release-activated calcium (CRAC) channels) in the plasma membrane. Store-operated Ca²⁺ entry (SOCE) is the process in which the emptying of Ca²⁺ stores itself activates Ca²⁺ channels in the plasma membrane to help refill the stores (Putney, Cell Calcium, 7, 1-12, 1986; Parekh et al, Physiol. Rev. 757-810; 2005). SOCE does more than simply provide Ca²⁺ for refilling stores, but itself generates sustained Ca²⁺ signals that control such essential functions as gene expression, cell metabolism and exocytosis (Parekh and Putney, Physiol. Rev. 85, 757-810 (2005).

In lymphocytes and mast cells, activation of antigen or Fc receptors causes the release of Ca²⁺ from intracellular stores, which in turn leads to Ca²⁺ influx through CRAC channels in the plasma membrane. The subsequent rise in intracellular Ca²⁺ activates calcineurin, a phosphatase that regulates the transcription factor NFAT. In resting cells, NFAT is phosphorylated and resides in the cytoplasm, but when dephosphorylated by calcineurin, NFAT translocates to the nucleus and activates different genetic programmes depending on stimulation conditions and cell type. In response to infections and during transplant rejection, NFAT partners with the transcription factor AP-1 (Fos-Jun) in the nucleus of “effector” T cells, thereby transactivating cytokine genes, genes that regulate T cell proliferation and other genes that orchestrate an active immune response (Rao et al., Annu Rev Immunol, 1997; 15:707-47). In contrast, in T cells recognizing self antigens, NFAT is activated in the absence of AP-1, and activates a transcriptional programme otherwise known as “anergy” that suppresses autoimmune responses (Macian et al., Transcriptional mechanisms underlying lymphocyte tolerance. Cell. 2002 Jun. 14; 109(6):719-31). In a subclass of T cells, known as regulatory T cells which suppress autoimmunity mediated by self-reactive effector T cells, NFAT partners with the transcription factor FOXP3 to activate genes responsible for suppressor function (Wu et al., Cell, 2006 Jul. 28; 126(2):375-87; Rudensky A Y, Gavin M, Zheng Y. Cell. 2006 Jul. 28; 126(2):253-256).

The endoplasmic reticulum (ER) carries out a variety processes. The ER has a role as both an agonist-sensitive Ca²⁺ store and sink, protein folding/processing takes place within its lumen. Here, numerous Ca²⁺-dependent chaperone proteins ensure that newly synthesized proteins are folded correctly and sent off to the appropriate destination. The ER is also involved in vesicle trafficking, release of stress signals, regulation of cholesterol metabolism, and apoptosis. Many of these processes require intraluminal Ca²⁺, and protein misfolding, ER stress responses, and apoptosis are all likely induced by depleting the ER of Ca²⁺ for prolonged periods of time. Because of its role as a source of Ca²⁺, it is clear that ER Ca²⁺ content must fall after stimulation. However, to preserve the functional integrity of the ER, it is vital that the Ca²⁺ content does not fall too low or is maintained at a low level. Replenishment of the ER with Ca²⁺ is therefore a central process to all eukaryotic cells. Because a fall in ER Ca²⁺ content activates store-operated Ca²⁺ channels in the plasma membrane, a major function of this Ca²⁺ entry pathway is believed to be maintenance of ER Ca²⁺ levels that are necessary for proper protein synthesis and folding. However, store-operated Ca²⁺ channels have other important roles.

The understanding of store operated calcium entry was provided by electrophysiological studies which established that the process of emptying the stores activated a Ca²⁺ current in mast cells called Ca²⁺ release-activated Ca²⁺ current or ICRAC. ICRAC is non-voltage activated, inwardly rectifying, and remarkably selective for Ca²⁺. It is found in several cell types mainly of hemopoietic origin. ICRAC is not the only store-operated current, and it is now apparent that store-operated influx encompasses a family of Ca²⁺-permeable channels, with different properties in different cell types. ICRAC was the first store-operated Ca²⁺ current to be described and remains a popular model for studying store-operated influx.

### Methods of Treatment and Uses

In the methods of treatment and uses described herein, one or more additional active agents can be administered with Compound (A) or a pharmaceutically acceptable salt thereof. For example, Compound (A) or a pharmaceutically acceptable salt thereof may be used in combination (administered together or sequentially) with one or more anti-cancer treatments such as, e.g., chemotherapy, radiation therapy, biological therapy, bone marrow transplantation, stem cell transplant or any other anticancer therapy, or one or more cytostatic, cytotoxic or anticancer agents or targeted therapy, either alone or in combination, such as, but not limited to, for example, DNA interactive agents, such as fludarabine, cisplatin, chlorambucil, bendamustine or doxorubicin; alkylating agents, such as cyclophosphamide; topoisomerase II inhibitors, such as etoposide; topoisomerase I inhibitors such as CPT-11 or topotecan; tubulin interacting agents, such as paclitaxel, docetaxel or the epothilones (for example ixabepilone), either naturally occurring or synthetic; hormonal agents, such as tamoxifen; thymidilate synthase inhibitors, such as 5-fluorouracil; and anti-metabolites, such as methotrexate; other tyrosine kinase inhibitors such as gefitinib (marketed as Iressac) and erlotinib (also known as OSI-774); angiogenesis inhibitors; EGF inhibitors; VEGF inhibitors; CDK inhibitors; SRC inhibitors; c-Kit inhibitors; Her1/2 inhibitors, checkpoint kinase inhibitors and monoclonal antibodies directed against growth factor receptors such as erbitux (EGF) and herceptin (Her2); CD20 monoclonal antibodies such as rituximab, ublixtumab (TGR-1101), ofatumumab (HuMax; Intracel), ocrelizumab, veltuzumab, GA101 (obinutuzumab), ocaratuzumab (AME-133v, LY2469298, Applied Molecular Evolution, Mentrik Biotech), PRO131921, tositumomab, veltuzumab (hA20, Immunomedics, Inc.), ibritumomab-tiuxetan, BLX-301 (Biolex Therapeutics), Reditux (Dr. Reddy's Laboratories), and PRO70769 (described in WO2004/056312); other B-cell targeting monoclonal antibodies such as belimumab, atacicept or fusion proteins such as blisibimod and BR3-Fc, other monoclonal antibodies such as alemtuzumab and other protein kinase modulators.

The methods of treatment and uses described herein also include use of one or more additional active agents to be administered with Compound (A), or a pharmaceutically acceptable salt, thereof. For example, CHOP (cyclophosphamide, doxorubicin, vincristine, prednisone); R-CHOP (rituximab-CHOP); hyperCV AD (hyperfractionated cyclophosphamide, vincristine, doxorubicin, dexamethasone, methotrexate, cytarabine); R-hyperCV AD (rituximab-hyperCV AD); FCM (fludarabine, cyclophosphamide, mitoxantrone); R-FCM (rituximab, fludarabine, cyclophosphamide, mitoxantrone); bortezomib and rituximab; temsirolimus and rituximab; temsirolimus and Velcade®; Iodine-131 tositumomab (Bexxar®) and CHOP; CVP (cyclophosphamide, vincristine, prednisone); R-CVP (rituximab-CVP); ICE (iphosphamide, carboplatin, etoposide); R-ICE (rituximab-ICE); FCR (fludarabine, cyclophosphamide, rituximab); FR (fludarabine, rituximab); and D.T. PACE (dexamethasone, thalidomide, cisplatin, adriamycin, cyclophosphamide, and etoposide).

The CRAC modulators, including Compound (A) and pharmaceutically acceptable salts thereof, may also be used in combination (administered together or sequentially) with one or more steroidal anti-inflammatory drugs, non-steroidal anti-inflammatory drugs (NSAIDs) or immune selective anti-inflammatory derivatives (ImSAIDs).

In one embodiment, the CRAC channel modulator, such as Compound (A) or a pharmaceutically acceptable salt thereof, can also be administered in combination with one or more other active principles useful in one of the pathologies mentioned above, for example an anti-emetic, analgesic, anti-inflammatory or anti-cachexia agent.

In another embodiment, the CRAC channel modulator, such as Compound (A) or a pharmaceutically acceptable salt thereof, can be combined with a radiation treatment.

In another embodiment, the CRAC channel modulator, such as Compound (A) or a pharmaceutically acceptable salt thereof, can be combined with surgery including either pre, post, or during period of surgery.

In any of the methods/uses described herein, the compositions described herein can be administered simultaneously, separately, sequentially and/or spaced in time.

### CRAC Modulators

The CRAC modulator may be any known in the art, such as those described in International Publication No. WO 11/042798 (including Compound (A) and pharmaceutically acceptable salts thereof), which is hereby incorporated by reference in its entirety. The CRAC modulators (such as Compound (A) or a pharmaceutically acceptable salt thereof) may inhibit store operated calcium entry, interrupt the assembly of SOCE units, alter the functional interactions of proteins that form store operated calcium channel complexes, and alter the functional interactions of STIM1 with Orai1. The CRAC channel modulators are SOC channel pore blockers, and are CRAC channel pore blockers.

The compounds described herein modulators (such as Compound (A) or a pharmaceutically acceptable salt thereof) modulate intracellular calcium and may be used in the treatment of diseases, disorders or conditions where modulation of intracellular calcium has a beneficial effect. In one embodiment, the compound of the present invention described herein inhibit store operated calcium entry. In one embodiment, the compounds of the present invention (such as Compound (A) or a pharmaceutically acceptable salt thereof) are capable of modulating intracellular calcium levels interrupt the assembly of SOCE units. In another embodiment, the compounds of the present invention (such as Compound (A) or a pharmaceutically acceptable salt thereof) are capable of modulating intracellular calcium levels alter the functional interactions of proteins that form store operated calcium channel complexes. In one embodiment, the compounds of the present invention (such as Compound (A) or a pharmaceutically acceptable salt thereof) are capable of modulating intracellular calcium levels alter the functional interactions of STIM1 with Orai1. In other embodiments, the compounds of the present invention (such as Compound (A) or a pharmaceutically acceptable salt thereof) are capable of modulating intracellular calcium levels are SOC channel pore blockers. In other embodiments, the compounds of the present invention (such as Compound (A) or a pharmaceutically acceptable salt thereof) are capable of modulating intracellular calcium levels are CRAC channel pore blockers.

In one aspect, the compounds of the present invention (such as Compound (A) or a pharmaceutically acceptable salt thereof) are capable of modulating intracellular calcium levels inhibit the electrophysiological current (ISOC) directly associated with activated SOC channels. In one aspect, the compounds of the present invention are capable of modulating intracellular calcium levels inhibit the electrophysiological current (ICRAC) directly associated with activated CRAC channels.

Compound (A) (N-[4-(3,5-dicyclopropyl-1H-pyrazol-1-yl)phenyl]-2-(quinolin-6-yl)acetamide) and pharmaceutically acceptable salts thereof (such as a hydrochloride salt) can be prepared as described in International Publication No. WO 11/042798.

Compound (A) and its salts modulate an activity of, modulate an interaction of, or bind to, or interact with at least one portion of a protein in the store operated calcium channel complex. In one embodiment, the compound of the present invention described herein modulate an activity of, modulate an interaction of, or bind to, or interact with at least one portion of a protein in the calcium release activated calcium channel complex. In one embodiment, the compounds of the present invention described herein reduce the level of functional store operated calcium channel complexes. In another embodiment, the compounds of the present invention described herein reduce the level of activated store operated calcium channel complexes. In a further embodiment, the store operated calcium channel complexes are calcium release activated calcium channel complexes.

### Pharmaceutical Compositions

The pharmaceutical compositions described herein may comprise a CRAC channel modulator (preferably a CRAC channel inhibitor, such as Compound (A) or a pharmaceutically acceptable salt thereof) and optionally one or more pharmaceutically acceptable carriers or excipients.

In one embodiment, the pharmaceutical composition includes a therapeutically effective amount of a CRAC channel modulator, such as Compound (A) or a pharmaceutically acceptable salt thereof. The pharmaceutical composition may include one or more additional active ingredients, as described herein.

Suitable pharmaceutical carriers and/or excipients may be selected from diluents, fillers, salts, disintegrants, binders, lubricants, glidants, wetting agents, controlled release matrices, colorants, flavorings, buffers, stabilizers, solubilizers, and any combination of any of the foregoing.

The pharmaceutical compositions described herein can be administered alone or in combination with one or more other active agents. Where desired, the CRAC channel modulator(s) and other agent(s) may be mixed into a preparation or both components may be formulated into separate preparations to use them in combination separately or at the same time.

The pharmaceutical compositions described herein can be administered together or in a sequential manner with one or more other active agents. Where desired, the CRAC channel modulator and other agent(s) may be co-administered or both components may be administered in a sequence to use them as a combination.

The CRAC channel modulator and pharmaceutical compositions described herein can be administered by any route that enables delivery of the CRAC channel modulator to the site of action, such as orally, intranasally, topically (e.g., transdermally), intraduodenally, parenterally (including intravenously, intraarterially, intramuscularally, intravascularally, intraperitoneally or by injection or infusion), intradermally, by intramammary, intrathecally, intraocularly, retrobulbarly, intrapulmonary (e.g., aerosolized drugs) or subcutaneously (including depot administration for long term release e.g., embedded-under the-splenic capsule, brain, or in the cornea), sublingually, anally, rectally, vaginally, or by surgical implantation (e.g., embedded under the splenic capsule, brain, or in the cornea).

The pharmaceutical compositions described herein can be administered in solid, semi-solid, liquid or gaseous form, or may be in dried powder, such as lyophilized form. The pharmaceutical composition can be packaged in forms convenient for delivery, including, for example, solid dosage forms such as capsules, sachets, cachets, gelatins, papers, tablets, suppositories, pellets, pills, troches, and lozenges. The type of packaging will generally depend on the desired route of administration. Implantable sustained release formulations are also contemplated, as are transdermal formulations.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages.

Oral solid dosage forms are described in, e.g., Remington's Pharmaceutical Sciences, 20th Ed., Lippincott Williams & Wilkins., 2000, Chapter 89, “Solid dosage forms include tablets, capsules, pills, troches or lozenges, and cachets or pellets”. Also, liposomal or proteinoid encapsulation may be used to formulate the compositions (as, for example, proteinoid microspheres reported in U.S. Pat. No. 4,925,673). Liposomal encapsulation may include liposomes that are derivatized with various polymers (e.g., U.S. Pat. No. 5,013,556). The pharmaceutical compositions described herein may include a CRAC channel modulator and inert ingredients which protect against degradation in the stomach and which permit release of the biologically active material in the intestine.

The amount of the CRAC channel modulator, such as Compound (A) or a pharmaceutically acceptable salt thereof, to be administered is dependent on the mammal being treated, the severity of the disorder or condition, the rate of administration, the disposition of the compound and the discretion of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.05 to about 2.5 g/day An effective amount of a compound of the invention may be administered in either single or multiple doses (e.g., two or three times a day).

The term “co-administration,” “administered in combination with,” and their grammatical equivalents, as used herein, encompasses administration of two or more agents to a subject so that both agents and/or their metabolites are present in the animal at the same time. Co-administration includes simultaneous administration in separate compositions, administration at different times in separate compositions, or administration in a composition in which both agents are present.

More preferably, the CRAC channel modulator is Compound (A) or a pharmaceutically acceptable salt thereof. In one preferred embodiment, Compound (A) is in the form of its hydrochloride salt (e.g., N-[4-(3,5-dicyclopropyl-1H-pyrazol-1-yl)phenyl]-2-(quinolin-6-yl)acetamide hydrochloride). For instance, in one embodiment, the pharmaceutical composition includes N-[4-(3,5-dicyclopropyl-1H-pyrazol-1-yl)phenyl]-2-(quinolin-6-yl)acetamide hydrochloride.

A further embodiment of the present invention relates to a method of treating haematological and solid cancers comprising administering a therapeutically effective amount of a pharmaceutical composition as described herein to a subject (preferably, a human subject) in need thereof.

A further embodiment of the present invention relates to the use of a pharmaceutical composition as described herein in the preparation of a medicament for treating haematological and solid cancers.

The following general methodology described herein provides the manner and process of using the CRAC channel modulator and are illustrative rather than limiting. Further modification of provided methodology and additionally new methods may also be devised in order to achieve and serve the purpose of the invention. Accordingly, it should be understood that there may be other embodiments which fall within the spirit and scope of the invention as defined by the specification hereto

### Routes of Administration

In any of the methods and uses described herein, the CRAC channel modulator and pharmaceutical composition may be administered by various routes. For example, the CRAC channel modulator and pharmaceutical composition may be formulated for injection, or for oral, nasal, transdermal or other forms of administration, including, e.g., by intravenous, intradermal, intramuscular, intramammary, intraperitoneal, intrathecal, intraocular, retrobulbar, intrapulmonary (e.g., aerosolized drugs) or subcutaneous injection (including depot administration for long term release e.g., embedded-under the-splenic capsule, brain, or in the cornea), by sublingual, anal, or vaginal administration, or by surgical implantation, e.g., embedded under the splenic capsule, brain, or in the cornea. The treatment may consist of a single dose or a plurality of doses over a period of time. In general, the uses and methods described herein involve administering an effective amount of a CRAC channel modulator (such as Compound (A) or a pharmaceutically acceptable salt thereof) together with one or more pharmaceutically acceptable diluents, preservatives, solubilizers, emulsifiers, adjuvants and/or carriers, as described above.

The present invention is now further illustrated by means of biological examples. In the examples below, Compound (A) was administered as its hydrochloride salt.

### EXAMPLES

### Biological Evaluation Illustrating the Effect of Compound (A) on Haematological and Solid Cancers

### Example 1

### Induction of Caspase 3 by Compound (A)

NCH-H460, A549 and NCI-H1975 cells were incubated with desired concentrations of the test compound for 48 hours. An equal number of cells per well (0.3×106 cells) were used. The increase in apoptosis manifested by an elevation in caspase-3 levels was determined using a Caspase-3 kit from Millipore. Induction of Caspase 3 by Compound (A) was measured fluorimetrically.

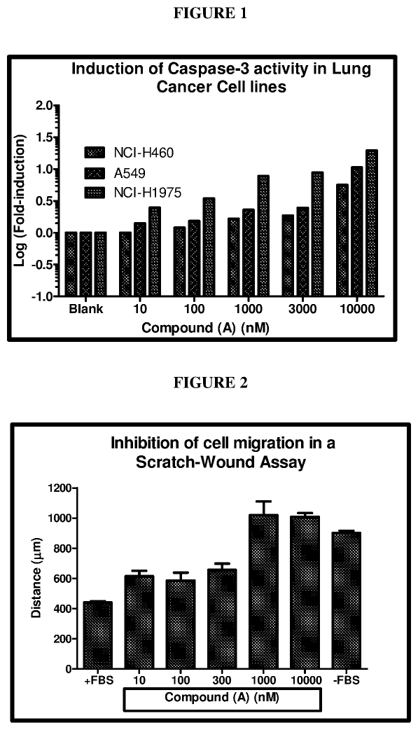

As can be seen in FIG. 1, a dose-dependent increase in caspase-3 was observed with Compound (A) in the cell lines tested (NCH-H460, A549 and NCI-H1975).

### Example 2

### Effect of Compound (A) on Migration in A549 Cells

A scratch was made to a serum-starved monolayer of A549 cells followed by washing and incubation with a desired concentration of Compound (A) in media with 10% fetal bovine serum (FBS) for 72 hours. The distance between the two edges of the wound was measured and percent inhibition was calculated with respect to a control.

As can be seen from FIG. 2, Compound (A) caused a dose-dependent reduction in FBS induced migration of A549 cells thereby implicating a role for this compound in the attenuation of metastasis.

### Example 3

### Anti-Proliferative Effect of Compound (A) in Various Cell Lines (MTT Assay)

A panel of cancer cell lines representing various different types of cancers were plated in 96-well plates and incubated with desired concentrations of Compound (A) for 48-72 hours. At the end of the incubation period, MTT ((3-(4,5-dimethylthiazol-2-yl)-2,5-diphenyltetrazolium bromide)) was added. The plates were placed on a shaker for 5 minutes to mix the formazan and the optical density at 560 nM was measured using a spectrophotometer. Data were plotted using Graphpad Prism for calculation of the GI₅₀ concentrations.

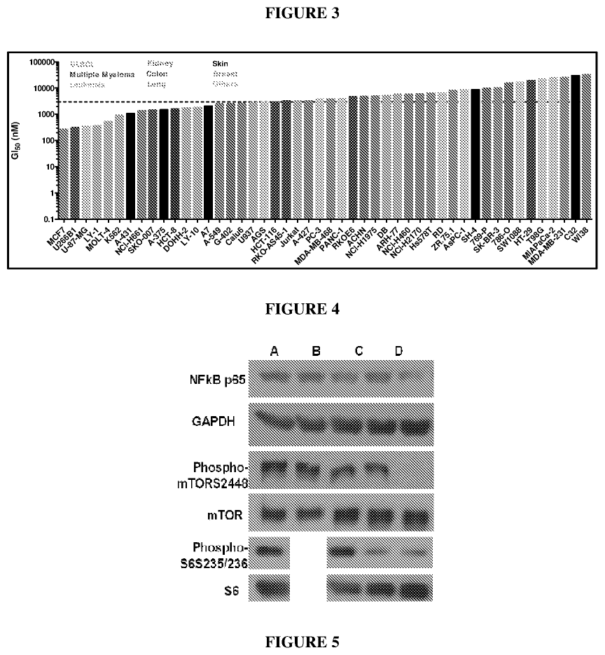

As can be seen from FIG. 3, the majority of the cell lines tested were sensitive to Compound (A), with GI₅₀ values ranging between 0.3 and 3 μM. Compound (A) did not affect growth of the normal lung cell line WI-38, indicating selectivity towards cancer cells.

### Example 4

### Effect of Compound (A) on Apoptosis in a Panel of Cell Lines

An antibody to activated caspase-3 was used to label cells from early to late stage apoptosis. The concentration of Compound (A) that caused a 2-fold induction in the caspase-3 signal was determined, indicating a significant apoptosis induction. The results are provided in Table 1 below.

TABLE 1
Apoptosis in Cell Lines Treated with Compound (A)

| | Apoptosis | | G1/S cell |
|---|---|---|---|
| | 2 Fold Induction | | cycle block |
| Cell Line | (μM) | Apoptosis Eₘₐₓ | (μM) |
| PC-3 | 2.05 | 12.31 | 8.13 |
| A7 | 2.22 | 55.74 | 2.78 |
| A427 | 2.77 | 16.1 | 3.52 |
| HCT-116 | 2.97 | 20.36 | 1.89 |
| MDA MB 468 | 3.02 | 8.86 | 7.81 |
| DOHH-2 | 3.03 | 6.13 | 3.23 |
| G-402 | 3.11 | 5.14 | 3.74 |
| RKO-AS45-1 | 3.15 | 40.99 | 3.94 |
| Calu6 | 3.73 | 3.34 | 10.5 |
| DB | 4.08 | 16.8 | 7.03 |
| RKOE6 | 4.64 | 14.43 | 4.85 |
| AGS | 5.04 | 26.62 | 3.16 |
| SKO-007 | 6.42 | 15.45 | 5.2 |
| PANC-1 | 8.83 | 11.49 | 7.32 |
| 786-O | 10.31 | 4.67 | 4.4 |
| U266B1 | 11.11 | 4.88 | 3.56 |
| ACHN | 11.23 | 2.86 | 10.3 |
| RD | 11.45 | 3.03 | 7.08 |
| SK-BR-3 | 11.6 | 8.32 | 6.11 |
| SH-4 | 15.59 | 72.67 | 16.6 |
| NCI-H661 | 17.64 | 13.23 | 2.8 |
| MDA MB 231 | 18.4 | 4.35 | N/A |
| 769-P | 24.71 | 10.3 | 4.82 |
| ARH-77 | 30.74 | 5.36 | 7.02 |
| T98G | 53.96 | 21.74 | 41.3 |
| SW1088 | 75.51 | 8.54 | 33 |
| C32 | 80.52 | 4.7 | 10.7 |
| Hs 578T | N/A | 1.19 | 4.56 |
| AsPC-1 | N/A | 2.07 | 3.64 |
| Wi38 | N/A | 0.49 | 8.82 |

### Example 5

### Inhibitory Effect of Compound (A) on NFkB, Phospho-mTOR and Phospho-S6 in Jurkat Cells

Cell lines were plated in RPMI media at a pre-determined density in a 6-well plate. After overnight incubation, cells were treated with Compound (A) for 24 hours. Cell lysates were analyzed for the expression of markers by Western Blotting. Gapdh, mTOR and S6 were used as a loading control for NFkB, phospho-mTOR, and phospho-S6 respectively. Lanes: A: Blank; B: 10 nM; C: 100 nM; D: 1000 nM.

As can be seen from FIG. 4, treatment with Compound (A) caused a dose-dependent reduction in NFkB, phospho-mTOR, phospho-S6 in Jurkat cells.

### Example 6

### The Anti-Tumor Effect of Compound (A) in NCI-H460 Human Non-Small Cell Lung Cancer Xenografts Model

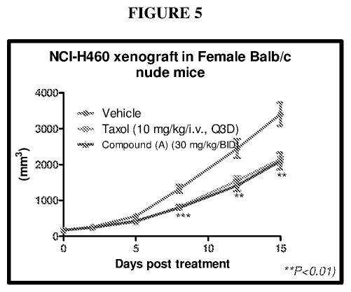

The anti-tumor effect of Compound (A) was determined as a single agent in a NCI-H460 human non-small cell lung cancer xenografts model using female Balb/c nude mice. 10⁶ cells were injected into the flank region. A week after tumor cell injection, mice either received the vehicle, oral administration of Compound (A) at 30 mg/kg BID or intravenous administration of taxol at 10 mg/kg Q3D (every three days) across a 15 day period. At the end of the study period, animals were sacrificed and the tumors harvested. The results are shown in FIG. 5.

As can be seen from FIG. 5, Compound (A) did not affect the mice body weight. Treatment with Compound (A) resulted in a significant inhibition of tumor growth in animals bearing NCI-H460 human non-small cell lung tumor xenografts. The tumor growth inhibition (TGI) value on day 15 was 36.7% for taxol and 38.3% for Compound (A).

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as described above. It is intended that the appended specification define the scope of the invention and that methods and structures within the scope of these specification and their equivalents be covered thereby.

All publications, patents and patent applications cited in this application are herein incorporated by reference to the same extent as if each individual publication, patent or patent application was specifically and individually indicated to be incorporated herein by reference.

1. A method of treating haematological cancer comprising administering to a subject a calcium release-activated calcium channel modulator.

2. A method of treating solid cancer comprising administering to a subject a calcium release-activated calcium channel modulator.

3. The method of claim 1, wherein the calcium release-activated calcium channel modulator is a calcium release-activated calcium channel inhibitor.

4. The method of claim 1, wherein the calcium release-activated calcium channel modulator is N-(4-(3,5-dicyclopropyl-1H-pyrazol-1-yl)phenyl)-2-(quinolin-6-yl)acetamide, or a pharmaceutically acceptable salt thereof.

5. The method of claim 1, wherein the calcium release-activated calcium channel modulator is a hydrochloride (HCl) salt of N-(4-(3,5-dicyclopropyl-1H-pyrazol-1-yl)phenyl)-2-(quinolin-6-yl) acetamide.

6. The method of claim 1, wherein the haematological cancer is leukemia, lymphoma or multiple myeloma.

7. The method of claim 2, wherein the solid cancer is sarcoma, carcinoma or lymphoma.

8. The method of claim 1, wherein the calcium release-activated calcium channel modulator is administered as a front-line therapy for the haematological cancer.

9. The method of claim 1, wherein the subject suffers from relapsed-refractory haematological cancer.

10. The method of claim 2, wherein the calcium release-activated calcium channel modulator is administered as a first-line therapy for the solid cancer.

11. The method of claim 2, wherein the subject suffers from non-resectable solid cancer.

12. The method of claim 1, wherein the subject is human.

13. (canceled)

14. The method of claim 1, wherein the calcium release-activated calcium channel modulator is administered by the oral route.

15. The method of claim 1, wherein the calcium release-activated calcium channel modulator is administered at a dose of
i) about 25 to about 1000 mg,
ii) about 25 to about 800 mg,
iii) about 25 to about 600 mg,
iv) about 25 to about 400 mg, or
v) about 25 to about 200 mg.

16. The method of claim 15, wherein the dose is
i) about 50 to about 1000 mg,
ii) about 50 to about 800 mg,
iii) about 50 to about 600 mg,
iv) about 50 to about 400 mg, or
v) about 50 to about 200 mg.

17. The method of claim 15, wherein the dose is
i) about 100 to about 1000 mg,
ii) about 100 to about 800 mg,
iii) about 100 to about 600 mg,
iv) about 100 to about 400 mg, or
v) about 100 to about 200 mg.

18. The method of claim 1, wherein the calcium release-activated calcium channel modulator is administered as a single or in divided doses.

19. The method of claim 1, wherein the calcium release-activated calcium channel modulator inhibits store operated calcium entry, interrupts the assembly of SOCE units, alters the functional interactions of proteins that form store operated calcium channel complexes, alters the functional interactions of STIM1 with Orai1, or any combination of any of the foregoing.

20. The method of claim 1, wherein the calcium release-activated calcium channel modulator is a SOC channel pore blocker or CRAC channel pore blocker.

21. The method of claim 1, wherein the calcium release-activated calcium channel modulator modulates intracellular calcium.

22. The method of claim 1, further comprising administering one or more anti-cancer treatments, one or more cytostatic, cytotoxic or anticancer agents, targeted therapy, or any combination of any of the foregoing.

23. The method of claim 22, wherein the calcium release-activated calcium channel modulator is administered together or sequentially with the one or more anti-cancer treatments, one or more cytostatic, cytotoxic or anticancer agents or targeted therapy.

24. The method of claim 22, wherein the anticancer agents are selected from DNA interactive agents, alkylating agents, topoisomerase II inhibitors, topoisomerase I inhibitors, tubulin interacting agents, hormonal agents, thymidilate synthase inhibitors, anti-metabolites, tyrosine kinase inhibitors, angiogenesis inhibitors, EGF inhibitors, VEGF inhibitors, CDK inhibitors, SRC inhibitors, c-Kit inhibitors, Her1/2 inhibitors, checkpoint kinase inhibitors, monoclonal antibodies directed against growth factor receptors selected from EGF and Her2, CD20 monoclonal antibodies, B-cell targeting monoclonal antibodies, fusion proteins, protein kinase modulators, CHOP (cyclophosphamide, doxorubicin, vincristine, prednisone), R-CHOP (rituximab-CHOP), hyperCV AD (hyperfractionated cyclophosphamide, vincristine, doxorubicin, dexamethasone, methotrexate, cytarabine), R-hyperCV AD (rituximab-hyperCV AD), FCM (fludarabine, cyclophosphamide, mitoxantrone), R-FCM (rituximab, fludarabine, cyclophosphamide, mitoxantrone), bortezomib and rituximab; temsirolimus and rituximab, temsirolimus and Velcade®, Iodine-131 tositumomab (Bexxar®) and CHOP, CVP (cyclophosphamide, vincristine, prednisone), R-CVP (rituximab-CVP), ICE (iphosphamide, carboplatin, etoposide), R-ICE (rituximab-ICE), FCR (fludarabine, cyclophosphamide, rituximab), FR (fludarabine, rituximab), and D.T. PACE (Dexamethasone, Thalidomide, Cisplatin, Adriamycin, Cyclophosphamide, Etoposide), steroidal anti-inflammatory drugs, non-steroidal anti-inflammatory drugs (NSAIDs), immune selective anti-inflammatory derivatives (ImSAIDs), anti-emetic, analgesic, anti-inflammatory, anti-cachexia agents, or any combination of any of the foregoing.

25. The method of claim 22, wherein the anticancer treatment is selected from chemotherapy, radiation therapy, biological therapy, bone marrow transplantation, stem cell transplant, or any combination of any of the foregoing.

26. A method of suppressing proliferation of solid cancer metastatic cells in a subject in need thereof, the method comprising administering to the subject a calcium release-activated calcium channel modulator.

27. The method of claim 26, wherein the calcium release-activated calcium channel modulator is a calcium release-activated calcium channel inhibitor.

28. The method of claim 26, wherein the calcium release-activated calcium channel modulator is N-(4-(3,5-dicyclopropyl-1H-pyrazol-1-yl)phenyl)-2-(quinolin-6-yl)acetamide, or a pharmaceutically acceptable salt thereof.

29-62. (canceled)

63. The method of claim 2, wherein the calcium release-activated calcium channel modulator is N-(4-(3,5-dicyclopropyl-1H-pyrazol-1-yl)phenyl)-2-(quinolin-6-yl)acetamide, or a pharmaceutically acceptable salt thereof.

64. The method of claim 2, wherein the calcium release-activated calcium channel modulator is a hydrochloride (HCl) salt of N-(4-(3,5-dicyclopropyl-1H-pyrazol-1-yl)phenyl)-2-(quinolin-6-yl)acetamide.
